(19) United States
(12) Patent Application Publication
(10) Pub. No.: US 2023/0333044 A1
(43) Pub. Date: Oct. 19, 2023

(54) SOLUTE-PHASE ELECTROCHEMICAL APTAMER SENSORS WITH RAPID TIME-TO-MEASUREMENT

(71) Applicant: University of Cincinnati, Cincinnati, OH (US)

(72) Inventor: Jason Heikenfeld, Cincinnati, OH (US)

(73) Assignee: University of Cincinnati, Cincinnati, OH (US)

(21) Appl. No.: 18/027,419

(22) PCT Filed: Sep. 24, 2021
(86) PCT No.: PCT/US2021/051862

§ 371 Date: Mar. 21, 2023

Related U.S. Application Data
(60) Provisional application No. 63/197,674, filed on Jun. 7, 2021.
(60) Provisional application No. 63/150,667, filed on Feb. 18, 2021.
(60) Provisional application No. 63/150,677, filed on Feb. 18, 2021.
(60) Provisional application No. 63/150,712, filed on Feb. 18, 2021.
(60) Provisional application No. 63/150,856, filed on Feb. 18, 2021.
(60) Provisional application No. 63/150,865, filed on Feb. 18, 2021.
(60) Provisional application No. 63/150,894, filed on Feb. 18, 2021.
(60) Provisional application No. 63/150,944, filed on Feb. 18, 2021.
(60) Provisional application No. 63/150,953, filed on Feb. 18, 2021.
(60) Provisional application No. 63/150,986, filed on Feb. 18, 2021.
(60) Provisional application No. 63/150,717, filed on Feb. 18, 2021.
(60) Provisional application No. 63/136,262, filed on Jan. 12, 2021.
(60) Provisional application No. 63/122,071, filed on Dec. 7, 2020.
(60) Provisional application No. 63/122,076, filed on Dec. 7, 2020.
(60) Provisional application No. 63/085,484, filed on Sep. 30, 2020.
(60) Provisional application No. 63/083,031, filed on Sep. 24, 2020.
(60) Provisional application No. 63/082,834, filed on Sep. 24, 2020.
(60) Provisional application No. 63/082,999, filed on Sep. 24, 2020.
(60) Provisional application No. 63/083,029, filed on Sep. 24, 2020.

(51) Int. Cl. G01N 27/327; G01N 33/543

(52) U.S. Cl. CPC G01N 27/3276; G01N 33/5438; G01N 27/3277; A61B 5/1468

(57) ABSTRACT

A device and method for detecting the presence of, or measuring the amount or concentration of, at least one analyte in a sample fluid, where the device includes a dissolvable material, and a plurality of aptamers disposed in and/or on the dissolvable material, and where one or more aptamers of the plurality of aptamers (1) are capable of binding at least one analyte, and (2) have at least one redox tag attached thereto.

### CROSS-REFERENCE TO RELATED APPLICATIONS

This application claims the benefit of the filing date of U.S. Patent Application Ser. No. 63/082,834, filed on Sep. 24, 2020; claims the benefit of the filing date of U.S. Patent Application Ser. No. 63/082,999, filed on Sep. 24, 2020; claims the benefit of the filing date of U.S. Patent Application Ser. No. 63/083,029, filed on Sep. 24, 2020; claims the benefit of the filing date of U.S. Patent Application Ser. No. 63/083,031, filed on Sep. 24, 2020; claims the benefit of the filing date of U.S. Patent Application Ser. No. 63/085,484, filed on Sep. 30, 2020; claims the benefit of the filing date of U.S. Patent Application Ser. No. 63/122,071, filed on Dec. 7, 2020; claims the benefit of the filing date of U.S. Patent Application Ser. No. 63/122,076, filed on Dec. 7, 2020; claims the benefit of the filing date of U.S. Patent Application Ser. No. 63/136,262, filed on Jan. 12, 2021; claims the benefit of the filing date of U.S. Patent Application Ser. No. 63/150,667, filed on Feb. 18, 2021; claims the benefit of the filing date of U.S. Patent Application Ser. No. 63/150,677, filed on Feb. 18, 2021; claims the benefit of the filing date of U.S. Patent Application Ser. No. 63/150,712, filed on Feb. 18, 2021; claims the benefit of the filing date of U.S. Patent Application Ser. No. 63/150,717, filed on Feb. 18, 2021; claims the benefit of the filing date of U.S. Patent Application Ser. No. 63/150,856, filed on Feb. 18, 2021; claims the benefit of the filing date of U.S. Patent Application Ser. No. 63/150,865, filed on Feb. 18, 2021; claims the benefit of the filing date of U.S. Patent Application Ser. No. 63/150,894, filed on Feb. 18, 2021; claims the benefit of the filing date of U.S. Patent Application Ser. No. 63/150,944, filed on Feb. 18, 2021; claims the benefit of the filing date of U.S. Patent Application Ser. No. 63/150,953, filed on Feb. 18, 2021; claims the benefit of the filing date of U.S. Patent Application Ser. No. 63/150,986, filed on Feb. 18, 2021; and claims the benefit of the filing date of U.S. Patent Application Ser. No. 63/197,674, filed on Jun. 7, 2021, the disclosures of each of which are incorporated by reference herein in their entireties.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Electrochemical aptamer sensors can identify the presence and/or concentration of an analyte of interest via the use of an aptamer sequence that specifically binds to the analyte of interest. These sensors include aptamers attached to an electrode, wherein each of the aptamers has a redox active molecule (redox couple) attached thereto. The redox couple can transfer electrical charge to or from the electrode. When an analyte binds to the aptamer, the aptamer changes shape, bringing the redox couple closer to or further from, on average, the electrode. This results in a measurable change in electrical current that can be translated to a measure of concentration of the analyte.

A major unresolved challenge for aptamer sensors (particularly those where the aptamers are bonded to the working electrode) is the lifetime of the sensors, especially for applications where continuous operation is required (“continuous” referring to multiple measurements over time by the same device). To date, it has been difficult to provide electrochemical aptamer sensors with a lifetime that allows continuous sensing to take place over an extended period of time. Furthermore, for aptamer sensors where the aptamer is bonded to the electrode, the flexibility of design is limited and often the sensitivity of the aptamer therefore suffers as a consequence.

Additionally, the ability to rapidly receive and accurate reading of the presence or concentration of an analyte may be important. In devices having aptamers that are free in solution, there are at least two challenges that increase the time before the device is ready to provide an accurate measurement: (1) the aptamers and any other solutes required for sensing must be in, or nearly in, a steady-state concentration across the sample fluid because measurement signal is proportional to concentration of the aptamers and redox tags; and (2) the thickness of any passivating layer or fouling layer on the electrode should not be rapidly changing during the measurement because otherwise electron transfer rates from redox tags are altered. To date, devices have been unable to reduce or eliminate these issues, And so, devices and methods that resolve these challenges for aptamer sensors are needed.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes.

One aspect of the present invention provides a device for detecting the presence of, or measuring the amount or concentration of, at least one analyte in a sample fluid—while resolving the challenges with current aptamer sensors described above. In this aspect, the device includes a dissolvable material, and a plurality of aptamers disposed in and/or on the dissolvable material. One or more aptamers of the plurality of aptamers (1) are capable of binding at least one analyte, and (2) have at least one redox tag attached thereto. The device further includes at least one electrode that is capable of detecting or measuring the at least one analyte through a change in electron transfer with the redox tags attached to the one or more aptamers.

The device may further include a volume (such as one that may be defined by one or more substrates) that is adapted to receive a sample fluid to be tested. The volume is in communicating relationship with the dissolvable material. And so, once sample fluid is introduced into the volume, the sample fluid will come into contact with the dissolvable material. This, in turn, releases one or more aptamers into the sample fluid where binding to any analyte in the sample fluid can occur. The electrode may be positioned a distance from the dissolvable material along a fluid path in the volume. This allows the aptamers (and any other solutes required for sensing) to be in, or nearly in, or approaching a steady-state concentration across the sample fluid by the time the sample fluid proceeds to the electrode for detection or measurement. The device then also has a time point at which the detection or measurement (of electron transfer—which correlates to analyte presence or concentration) occurs. This time point is subsequent to introduction of the sample fluid into the device.

In another aspect, the present invention provides a method of detecting the presence of, or measuring the concentration or amount of, an analyte in a sample fluid. The method includes introducing a sample fluid to a volume having a dissolvable material and at least one electrode therein, wherein the dissolvable material comprises a plurality of aptamers having a plurality of redox tags attached thereto. Thereafter, the method includes measuring the analyte based on a change in electron transfer between one or more redox tags of the plurality of redox tags and the electrode. This measuring may occur at a time point and, as described above, the time point is subsequent to introduction of the sample fluid into the fluid path.

### BRIEF DESCRIPTION OF THE DRAWINGS

The objects and advantages of the disclosed invention will be further appreciated in light of the following detailed descriptions and drawings in which:

FIG. 1A is a cross-sectional view of a device in accordance with principles of the disclosed invention.

FIG. 1B is a cross-sectional view of an alternate embodiment of a device in accordance with principles of the disclosed invention.

FIG. 2A is a schematic showing a prior art portion of an aptamer sensor device having a passivating layer and an aptamer attached to an electrode.

FIG. 2B is a schematic showing the aptamer and passivating layer portions of the aptamer sensor device of FIG. 2A degrading over time.

FIG. 3 is a schematic of yet another embodiment of a device in accordance with principles of the present invention.

FIG. 4 is a graph showing the effect of a membrane in an aptamer sensor device on percentage of solute retention versus molecular weight of the solute.

FIG. 5A is a schematic of yet another embodiment of a device in accordance with principles of the present invention.

FIG. 5B is a schematic showing an alternate embodiment of an aptamer with attached redox tag that can be used with devices in accordance with principles of the disclosed invention.

FIG. 5C is a schematic showing yet another alternate embodiment of an aptamer with attached redox tag that can be used with devices in accordance with principles of the disclosed invention.

FIG. 5D is a schematic showing yet another alternate embodiment of an aptamer with attached redox tag that can be used with devices in accordance with principles of the disclosed invention.

FIG. 5E is a schematic showing yet another alternate embodiment of an aptamer with attached redox tag that can be used with devices in accordance with principles of the disclosed invention.

FIG. 6A is a schematic of yet another embodiment of a device in accordance with principles of the present invention.

FIG. 6B is a schematic showing an alternate embodiment of an aptamer with attached redox tag that can be used with devices in accordance with principles of the disclosed invention.

FIG. 6C is a schematic showing yet another alternate embodiment of an aptamer with attached redox tag that can be used with devices in accordance with principles of the disclosed invention.

FIG. 7 is a schematic of yet another embodiment of a device in accordance with principles of the present invention.

FIG. 8 is a schematic of yet another embodiment of a device in accordance with principles of the present invention.

FIG. 9A is a cross-sectional view of a device according to an embodiment of the disclosed invention.

FIG. 9B is a cross-sectional view of a device according to an embodiment of the disclosed invention.

FIG. 10A is a graph showing raw chronoamperometric scans of current versus time for cortisol in an exemplary device.

FIG. 10B is a graph showing normalized current gain for three sensors versus concentration of cortisol.

### DEFINITIONS

As used herein, “continuous sensing” with a “continuous sensor” means a sensor that changes in response to changing concentration of at least one solute in a solution such as an analyte. Similarly, as used herein, “continuous monitoring” means the capability of a device to provide multiple measurements of an analyte over time.

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

As used herein, the term “electrode” means any material that is electrically conductive such as gold, platinum, nickel, silicon, conductive liquid infused materials such as ionic liquids, PEDOT:PSS, conductive oxides, carbon, boron-doped diamond, nanotubes or nanowire meshes, or other suitable electrically conducting materials.

As used herein, the term “blocking layer” or “passivating layer” means a homogeneous or heterogeneous layer of molecules on an electrode which alter the electrochemical behavior of the electrode. Examples include a monolayer of mercaptohexanol on a gold electrode or as another example natural small-molecule solutes in serum that form a layer on a carbon electrode.

As used herein, the term “aptamer” means a molecule that undergoes a conformation or binding change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function, but which behave analogous to traditional aptamers. Two or more aptamers bound together can also be referred to as an aptamer (i.e., not separated in solution). Aptamers can have molecular weights of at least 1 kDa, 10 kDa, or 100 kDa.

As used herein, the term “redox tag” or “redox molecule” means any species such as small or large molecules with a redox active portion that when brought adjacent to an electrode can reversibly transfer at least one electron with the electrode. Redox tag or molecule examples include methylene blue, ferrocene, quinones, or other suitable species that satisfy the definition of a redox tag or molecule. In some cases, a redox tag or molecule is referred to as a redox mediator. Redox tags or molecules may also exchange electrons with other redox tags or molecules.

As used herein, the term “change in electron transfer” means a redox tag whose electron transfer with an electrode has changed in a measurable manner This change in electron transfer can, for example, originate from availability for electron transfer, distance from an electrode, diffusion rate to or from an electrode, a shift or increase or decrease in electrochemical activity of the redox tag, or any other embodiment as taught herein that results in a measurable change in electron transfer between the redox tag and the electrode.

As used herein, the term “optical tag” or “fluorescent tag” means any species that fluoresces in response to an optical source such as LED and whose fluorescence is detectable by a photodetector such as a photodiode. Example fluorescent tags include fluorescein and may be used in combination with other fluorescent tags or optical quenchers such a black-hole quencher dyes to change the fluorescence of the optical tag.

As used herein, the term “signaling aptamer” means an aptamer that is tagged with a redox active molecule or tag and/or contains a redox active portion itself and which provides a change in electrochemical signal when it is released from an anchor aptamer.

As used herein, the term “anchor aptamer” means an aptamer that that can bind to a signaling aptamer, and when bound to the signaling aptamer changes at least one property of the bound vs. unbound signaling aptamer such as molecular weight, diffusion coefficient, charge state, being floating in solution vs. being immobilized, or some other property which achieves the stated effect for the signaling aptamer. The binding of the anchor aptamer with the signaling aptamer is dependent on concentration of the analyte to be measured.

As used herein, the term “folded aptamer” means an aptamer that along its length associates with itself in one or more locations creating a three-dimensional structure for the aptamer that is distinct from an “unfolded aptamer” that is a freely floating and oscillating strand of aptamer. Aptamers can also be partially folded or partially unfolded in structure or in time spent in the folded vs. unfolded states. Multiple folding configurations are also possible.

As used herein, the term “analyte” means any solute in a solution or fluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a solution or fluid.

As used herein, the term “membrane” means a polymer film, plug of hydrogel, liquid-infused film, tiny pore, or other suitable material which is permiselective to transport of a solute through the membrane by solute parameters such as size, charge state, hydrophobicity, physical structure, or other solute parameters than can enable permiselectivity. For example, a dialysis membrane is permselective by passing small solutes but not large solutes such as proteins. Membranes as understood herein need not be multiporous, for example a nanotube or nanopore can act as a permiselective filter and is therefore considered part of a membrane as understood for the present invention.

As used herein, the term “sample fluid” means any solution or fluid that contains at least one analyte to be measured.

As used herein, the term “sensor fluid” means a solution or fluid that differs from a sample solution by at least one property, and through which the sensor solution and the sample solution are therefore separated but are in fluidic connection through at least one pathway such as a membrane. The sensor solution comprises at least one aptamer as a solute.

As used herein, the term “reservoir fluid” means a solution or fluid that differs from a sample solution by at least one property, and through which the sensor solution and the reservoir solution are in fluidic connection through at least one pathway such as a membrane or a pin-hole connection. A reservoir fluid may have multiple function in a device, for example, by introducing a solute continuously or as needed by diffusion equilibrium into the sensor fluid, or for example removing unwanted solutes from a sensor fluid and acting as a “waste removal element”.

As used herein, a “device” comprises at least one sensor based on at least one aptamer, at least one sensor solution, and at least one sample solution. Devices can sense multiple samples and be in multiple configurations such as a device to measure a pin-prick of blood, or a microneedle or in-dwelling sensor needle to measure interstitial fluid, or a device to measure saliva, tears, sweat, or urine sensor, or a device to measure water pollutants or food processing solutes, or other devices which measure at least one analyte found in a sample solution.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors are preferably electrical in nature, but may also include optical such as a LED or laser excitation source and a photodetector, chemical, mechanical, or other known biosensing mechanisms. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a battery, antenna, adhesive), and for purposes of brevity and focus on inventive aspects, such components may not be explicitly shown in the diagrams or described in the embodiments of the disclosed invention. All ranges of parameters disclosed herein include the endpoints of the ranges.

Embodiments of the disclosed invention are directed to a sensing device for at least one analyte. The sensing device has a sample fluid with at least one analyte and at least one electrode that measures the analyte through a change in electron transfer for a plurality of redox tags attached to aptamers that are freely diffusing in fluid. In addition, the measurement has a time point, and the time point begins after the sample fluid is introduced to the device. In one embodiment, the time point is less than 20 minutes. In another embodiment, a method of measuring an analyte in a sample fluid is provided. The method involves a) exposing a sensing device to said sample fluid; and b) measuring the analyte for a time period. The time period begins after the sample fluid is introduced to the sensing device. In addition, the time period is less than 20 minutes. Further, the sensing device includes aptamers that are freely diffusing in fluid, the aptamers having a plurality of redox tags attached thereto. Also, the sensing device includes at least one electrode that measures the analyte through a change in electron transfer for said redox tags.

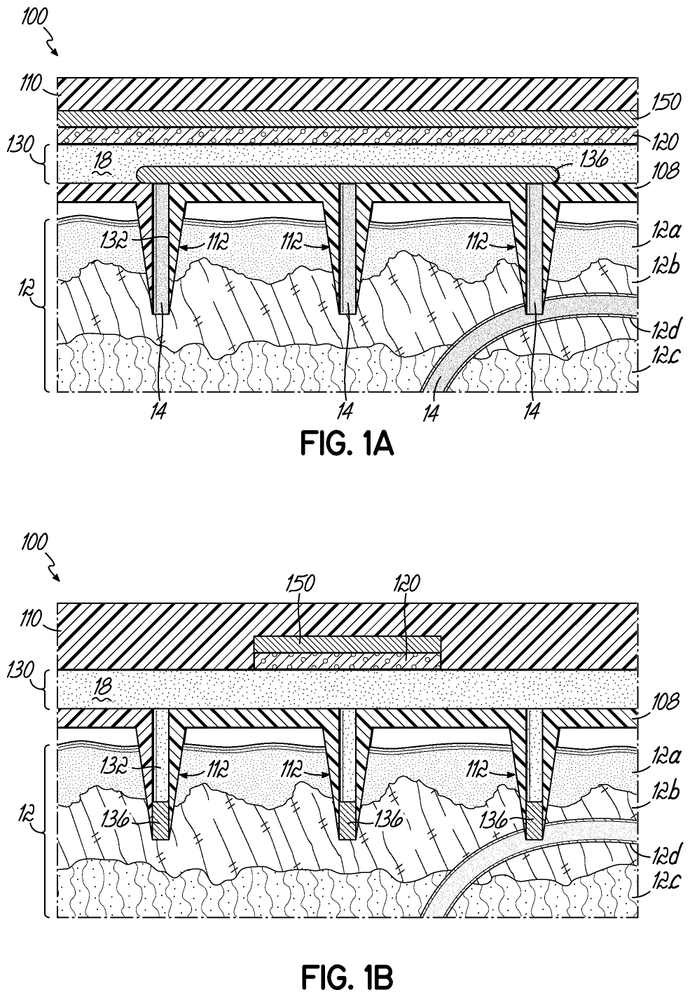

With reference to FIGS. 1A and 1B, exemplary embodiments of devices in accordance with principles of the disclosed invention are shown. Referring first to FIG. 1A, a device 100 is shown as being placed partially in-vivo into the skin 12 of a subject. Skin 12 includes the epidermis 12a, the dermis 12b, and the subcutaneous or hypodermis 12c. The device 100 includes a feature 112 that allows for access to sample fluids from the subject. Such sample fluids may include interstitial fluid (from the dermis 12b) and/or blood (from a capillary 12d). In the embodiment shown in FIG. 1A, the feature 112 includes a plurality of microneedles (which may be formed of metal, polymer, semiconductor, glass, or other suitable material). Each of the microneedles 112 projects from a first substrate 108. And each microneedle 112 may include a hollow lumen 132. The device 100 also includes a second substrate 110 (which may be a material such as polymer or glass) having an electrode 150 adjacent thereto. An optional passivating layer 120 may be adjacent to electrode 150, such that electrode 150 is positioned between passivating layer 120 and second substrate 110. Passivating layer 120 includes a compound such as mercaptohexanol or may comprise natural solutes that have diffused into the device 100 from the dermis 12b.

As can be seen in FIG. 1A, a defined volume 130 is present between first substrate 108 and passivating layer 120. It will be recognized by those of ordinary skill in the art that defined volume 130 does not necessarily have to be defined by first substrate 108 and passivating layer 120—and in embodiments where passivating layer is absent, volume 130 may be defined by first substrate 108 and electrode 150; or, alternatively, may be defined by first substrate 108 and second substrate 110. A sensor fluid 18 may be present within volume 130 (as shown in FIG. 1A). Further, as can be seen in the embodiment of FIG. 1A, at least one membrane 136 is present between first substrate 108 and passivating layer 120, and is positioned adjacent first substrate 108. The at least one membrane 136 may be of various materials or substances—such as a dialysis membrane or hydrogel, for example. In the particular embodiment shown in FIG. 1A, portions of the membrane 136 overlie the boundary between volume 130 and lumens 132 of each microneedle 112. Due to this positioning of membrane 136, volume 130 includes sensor fluid 18, and lumens 132 include sample fluid 14—such as interstitial fluid from dermis 12b or blood from capillary 12d. Together the total volume provided by volume 130 and lumens 132 can be a microfluidic component such as channels, a hydrogel, or other suitable material. A diffusion or other fluidic pathway exists from the sample fluid 14, such as interstitial fluid or blood, into volumes 132, 130.

Another embodiment of a device 100 is shown in FIG. 1B. This embodiment also includes first and second substrates 108, 110, microneedles 112 having lumens 132, electrode 150, passivating layer 120, defined volume 130 and at least one membrane 136. As can be seen from FIG. 1B, in this embodiment, electrode 150 and passivating layer 120 are recessed in second substrate 110 (as opposed to the configuration shown in FIG. 1A). Thus, volume 130 is defined by first substrate 108 and combination of second substrate 110 and passivating layer 120. Further, the embodiment as shown in FIG. 1B includes a plurality of membranes 136, with each membrane 136 positioned in a distal end of each lumen 132 of each microneedle 112. Due to this positioning of membranes 136, both volume 130 and lumens 132 include a sensor fluid 18 (and sample fluid is present in, and obtainable from, dermis 12b and capillary 12d, for example).

Alternative arrangements and materials to those discussed above with respect to FIGS. 1A and 1B are possible, such as using a single needle or hydrogel polymer microneedles. In addition, one or more of the features of device 100 or the entire device 100 could be implanted into the body and perform similarly as described herein. Furthermore, a device 100 could be fully outside the body, if for example sampling a fluid such as sweat or tears.

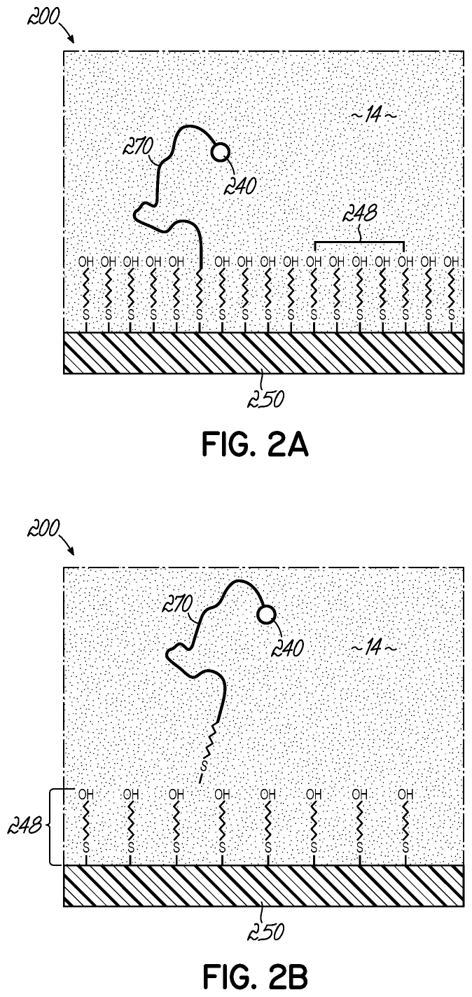

Turning now to FIGS. 2A and 2B, where like numerals refer to like features, a portion of a prior art device 200 is shown. Referring to FIG. 2A, an aptamer sensor includes a passivating or blocking layer 248 (including a compound such as mercaptohexanol) attached to an electrode 250 (made from a material such as gold), and having at least one aptamer 270 that is attached to the electrode 250, such as by being thiol-bonded to the electrode 250. The aptamer 270 has at least one redox tag or molecule 240, such as methylene blue, associated therewith. The device 200 is shown as being positioned in a sample fluid 14, such as blood or interstitial fluid (for example). This prior art device 200 may have an analyte (not shown) that binds with the aptamer 270, thereby changing the availability of the redox tag 240 to the electrode 250, such as by bringing it closer to, or further from, the electrode 250. Conventional aptamer sensors can be limited in performance because an aptamer that is bound to an electrode often has a weaker binding affinity to an analyte than an aptamer that is free in solution. In addition, as shown in FIG. 2B, the sensors can degrade as the aptamer 270 and/or blocking layer 248 degrades over time (e.g., chemical degradation, or detaching from the electrode 250). Also, because such prior art devices 200 have relied on exogenous molecules (e.g., mercaptohexanol) for passivation, the passivation layer 248 can also become thicker with fouling from solutes (such as albumin) in the sample fluid 14.

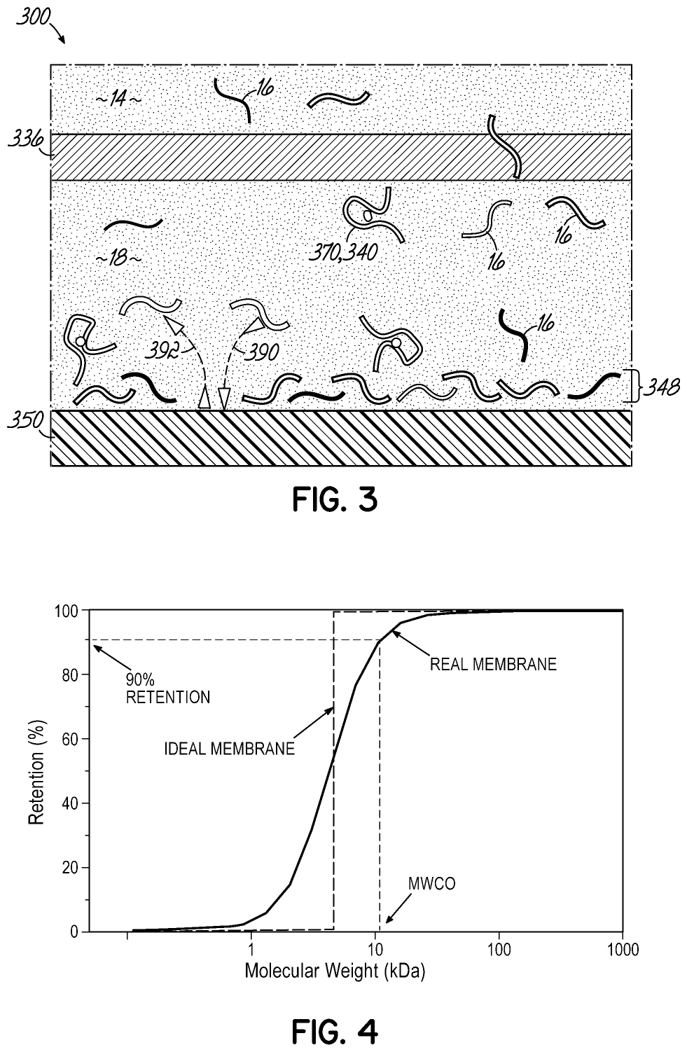

Thus, and with reference now to FIG. 3, where like numerals refer to like features, an embodiment of the disclosed invention that improves on the prior art devices and reduces or eliminates drawbacks with such devices is shown. To that end, FIG. 3 shows a device 300 (or at least a portion thereof) that includes an electrode 350 and at least one membrane 336 which separates a sample fluid 14 from a sensor fluid 18. The sensor fluid 18 contains a plurality of aptamers 370 having redox tags 340. The electrode 350 may include a passivating layer 348. The passivating layer 348 may comprise one or more endogeneous solutes 16 from the sample fluid 14 itself (or, as initially prepared, the passivating layer 348 may be prepared from molecules that are known to be endogenous to the sample fluid to be tested). Examples of such endogenous molecules 16 include small molecules such as amino acids, hormones, metabolites, or peptides. (Thus, the device 300 shown in FIG. 3 differs from that described above in prior art FIGS. 2A and 2B in that the prior art device described above included an aptamer and an exogenous molecule, such as mercaptohexanol. Similarly, electrode 350 could also contain a passivation layer 348 comprised, at least in part, by an exogenous molecule such as hexanethiol or mercaptohexanol. But, even in that case, the passivation layer could detatch from the electrode 350 and be in need of replacement.) By including endogeneous molecules 16 in the passivation layer 348, longer lifetime of the device 300 is achieved because endogenous molecules 16 can leave the electrode 350 as shown by arrow 392 and another endogenous molecule 16 can replace that now-missing molecule as shown by arrow 390. Thus, in a sense, the very molecules in the sample fluid 14 can be used to “repair” the passivation layer as it degrades, thereby extending the life of the device. (As mentioned above, these endogenous molecules can originate from the sample fluid itself, be already present as a deliberate component of the sensor fluid, or could be a mix of the two.) As a non-limiting example, membrane 336 is able to pass in small solutes (e.g., <1 kDa)—for example, an analyte such as cortisol—and passivating solutes 16, such as amino-acids and peptides, but retains the aptamer 370 (with redox tag 340) which is often >10 kDa in molecular weight. If the aptamer 370 with redox tag 340 were not retained by the membrane 336, then aptamer 370 with redox tag 340 could be lost into the body and no longer able be able to provide a measurement of the analyte.

An example of the analysis of the use of a membrane to pass small solutes (small target analyte) while retaining aptamers within device is shown with reference to FIG. 4, which shows an illustrative plot of solute retention for a membrane such as membrane 336. This is an example only, and shows that if measuring a small analyte such as cortisol (<400 Da) and using a large aptamer (>10 kDa or even >50 kDa) a membrane could be highly permeable to the analyte and poorly permeable to the aptamer. Thus, for example, in various embodiments, membranes of the present invention may have molecular weight cutoffs (i.e., the molecular weight above which a molecule will not easily pass through the membrane) that are at least one of <300 Da, <1000 Da, <3 kDa, <10 kDa, <30 kDa, <100 kDa, <300 kDa. Larger molecular weight cut-off membranes will require larger sized aptamers to prevent the aptamers from potentially escaping the device.

Several additional embodiments will be discussed below. In these additional embodiments, an increase in availability of the redox tag to the electrode can occur as a result of aptamer binding analyte, or, alternatively, without aptamer binding to an analyte. And even though each of the embodiments discussed below (and their respective figures) may show one specific example, the other the embodiments of the invention are not so limited (e.g., the various aptamer/redox tag types can be used across the various embodiments of devices disclosed herein, and vice versa.

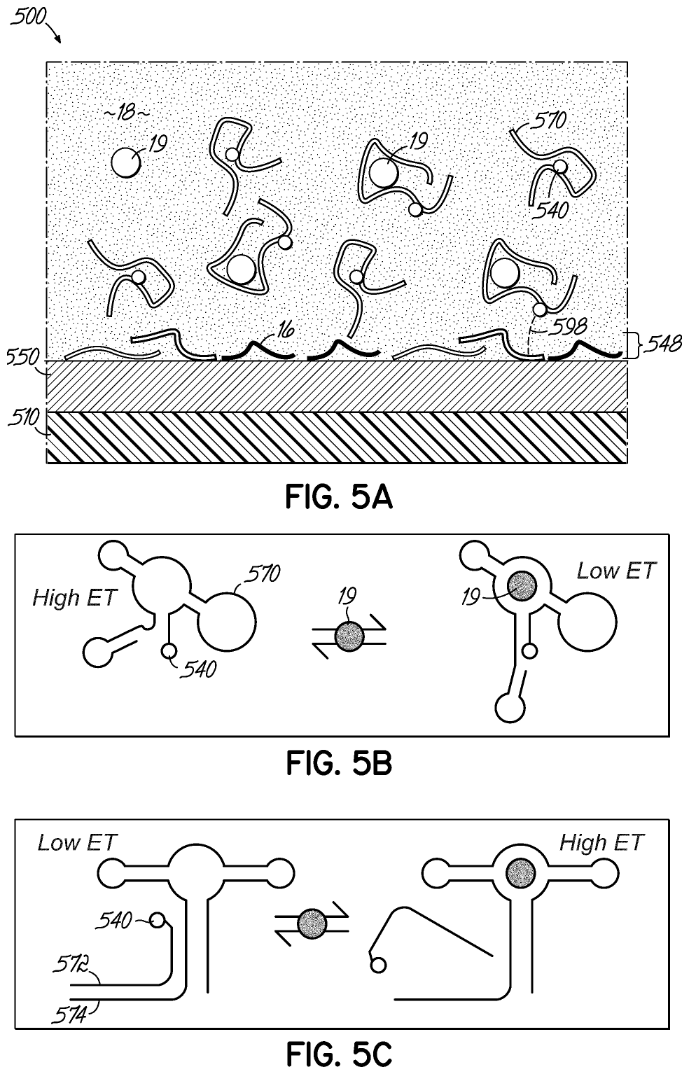

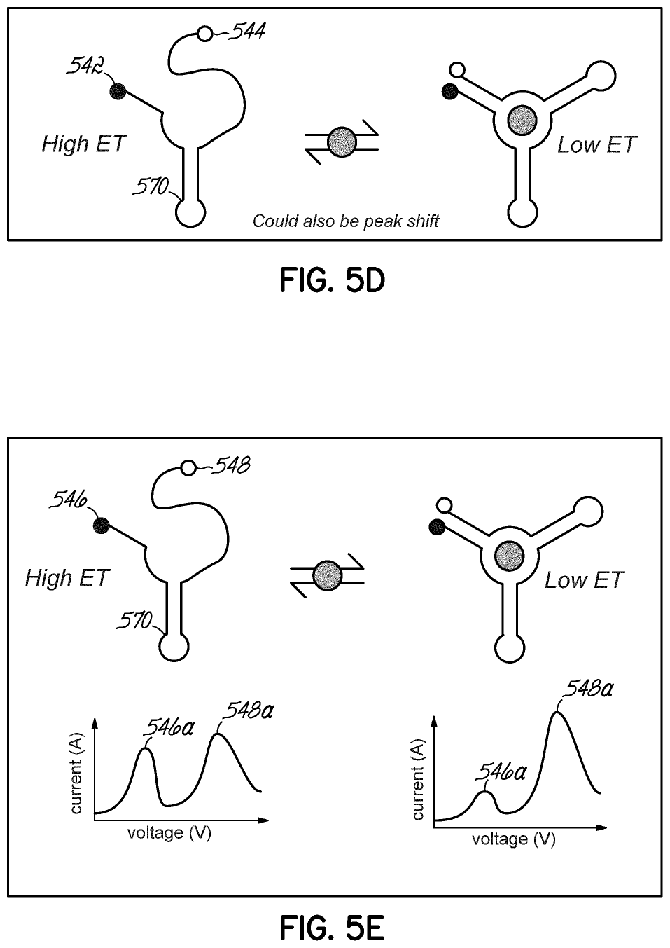

Turning now to FIGS. 5A-5E, where like numerals refer to like features: FIG. 5A shows a portion of a device 500 including a substrate 510, a sensor fluid 18, a plurality of aptamers 570 with redox tags 540 free in the sensor fluid 18, a passivation layer 548 of endogenous solutes 16, and an electrode adjacent the substrate 510. Though not part of the device, analytes 19 are also depicted as present in the sensor fluid of the device. The schematic shown in FIG. 5A also depicts an electron transfer event that occurs between a redox tag 540 and the electrode 550. This is shown generally at reference numeral 598, and is a non-limiting example depicting that electron transfer 598 from a redox tag 540 occurs in an increased amount, or frequency, or rate, when aptamer 570 binding to analyte 19 occurs (e.g., as shown in the figure, when analyte is not bound to aptamer, the redox tag is not available—or is less available—to the electrode, due to, for example, a conformation of the aptamer that hinders or prevents such transfer when not bound to analyte; conversely, when aptamer binds analyte, the conformation of aptamer may change in a manner that positions the redox tag for electron transfer). In various embodiments, aptamer binding to analyte can provide changes in electron transfer and redox current (compared to baseline transfer and current—i.e., transfer/current in the absence of analyte binding) of greater than 5%, greater than 10%, greater than 20%, greater than 50%, greater than 100%, or greater than 200%. For the embodiments illustrated herein, non-limiting examples of electrical measurement techniques may include voltammetry, square wave voltammetry, amperometry, chronoamperometry, coulometry, chronocoulometry, with a preferred embodiment being square wave voltammetry.

FIG. 5B schematically depicts another example of an aptamer 570 with attached redox tag 540 (that differs from the aptamer 570/redox tag 540 schematically shown in FIG. 5A). The embodiment of the aptamer 570 in FIG. 5B is designed such that the redox tag 540 is more available for electron transfer with the electrode 550 in the absence of any analyte 19 binding to the aptamer 570 (high electron transfer—or high ET). Conversely, when analyte 19 binds to the aptamer 570 of FIG. 5B, the redox tag 540 is less available for electron transfer with the electrode 550 (e.g., the redox tag 540 is less exposed—low ET).

FIG. 5C schematically depicts yet another example of an aptamer with attached redox tag 540 (that differs from the aptamer 570/redox tag 540 schematically shown in FIGS. 5A and 5B). The embodiment shown in FIG. 5C is designed with two aptamer portions: a signaling aptamer 572 and an anchor aptamer 574. A redox tag 540 is associated with (such as by being attached to) the signaling aptamer 572. The anchor aptamer 574 includes a portion that has affinity for, and thus can bind, analyte 19. When analyte 19 is not bound to the anchor aptamer 574 (left side of FIG. 5C), the signaling aptamer 572 remains associated with the anchor aptamer 574, and so the redox tag 540 on signaling aptamer 572 is less available for electron transfer with the electrode 550 (low ET). However, once the anchor aptamer 574 binds to analyte 19 (right side of FIG. 5C), signaling aptamer 572 is released from anchor aptamer 574, and the redox tag 540 becomes more available for electron transfer with the electrode 550 (high ET). It will be recognized that the device of the embodiment of FIG. 5C has a plurality of aptamers—and thus includes a plurality of signaling aptamers 572, and a plurality of anchor aptamers 574. As described above, each anchor aptamer of the plurality of anchor aptamers is adapted to bind to analyte. In one embodiment, each signaling aptamer of a majority of the plurality of signaling aptamers is bound to a respective anchor aptamer when a majority of anchor aptamers are not bound to any analyte. (This may occur, for example, prior to the introduction of any analyte.) Once analyte is introduced (such as when a sample fluid is introduced into the device—e.g., by being introduced into the sensor fluid of the device), at least a subset of anchor aptamers from the plurality of anchor aptamers then binds to analyte. When this occurs, a subset of signaling aptamers (from the total plurality of aptamers) dissociates from the anchor aptamers—and the redox tag becomes more available for electron transfer with the electrode.

Further, while the embodiment shown in FIG. 5C depicts analyte 19 binding to anchor aptamer 574 and redox tag 540 on signaling aptamer 572, in an alternate embodiment analyte binding may occur with signaling aptamer (signaling aptamer having redox tag), and binding of analyte to signaling aptamer may serve to release signaling aptamer from anchor aptamer (such as by change in conformation of signaling aptamer). Even further configurations as possible, as understood by those skilled in the art of aptamers. To maximize the signal gain (change in signal) signaling aptamer 572 concentration will typically be less than or equal to the anchor aptamer 574 concentration else the signaling aptamer can cause increased background signal with or without the presence of analyte.

FIG. 5D schematically depicts yet another example of an aptamer with attached redox tag 540 (that differs from the aptamers/redox tags schematically shown in FIGS. 5A, 5B, and 5C). The embodiment of the aptamer 570 in FIG. 5D has both a redox tag 544 and a redox quencher 542 associated therewith (such as by being bound to the aptamer 570). When analyte 19 is not bound to the aptamer 570, the redox tag 544 and redox quencher 542 are spatially separated (left side of FIG. 5D) thereby allowing for greater electron transfer between redox tag 544 and electrode 550 (high ET). However, once the aptamer 570 binds to analyte 19 (right side of FIG. 5D), the redox tag 544 and redox quencher 542 are brought into closer proximity with one another, thereby causing less electron transfer between redox tag 544 and electrode 550 (low ET). Numerous quenchers are possible, including anthraquinone-based redox molecules that can be self-quenching when two of such identical molecules are brought close together (monomer vs. dimer).

FIG. 5E schematically depicts yet another example of an aptamer with attached redox tag 540 (that differs from the aptamers/redox tags schematically shown in FIGS. 5A, 5B, 5C, and 5D). The embodiment of the aptamer 570 in FIG. 5E has both a first redox tag 546 and a second redox tag 548 associated therewith (such as by being bound to the aptamer 570). When analyte 19 is not bound to the aptamer 570, the first and second redox tags 546, 548 are spatially separated (left side of FIG. 5E) thereby allowing for greater electron transfer between first and second redox tags 546, 548 and electrode 550 (high ET). However, once the aptamer 570 binds to analyte 19 (right side of FIG. 5E), the first and second redox tags 546, 548 are brought closer together and the electron transfer from one of the redox tags 546, 548 to the electrode 550 is altered due to a two-step mediated electron transfer process, or other effect, for two redox tags brought into close proximity These changes in electron transfer are depicted in the voltammograms as shown as 546a and 548a. A non-limiting example of redox tags that enable the embodiment of FIG. 5E include methylene blue and ferricyanide.

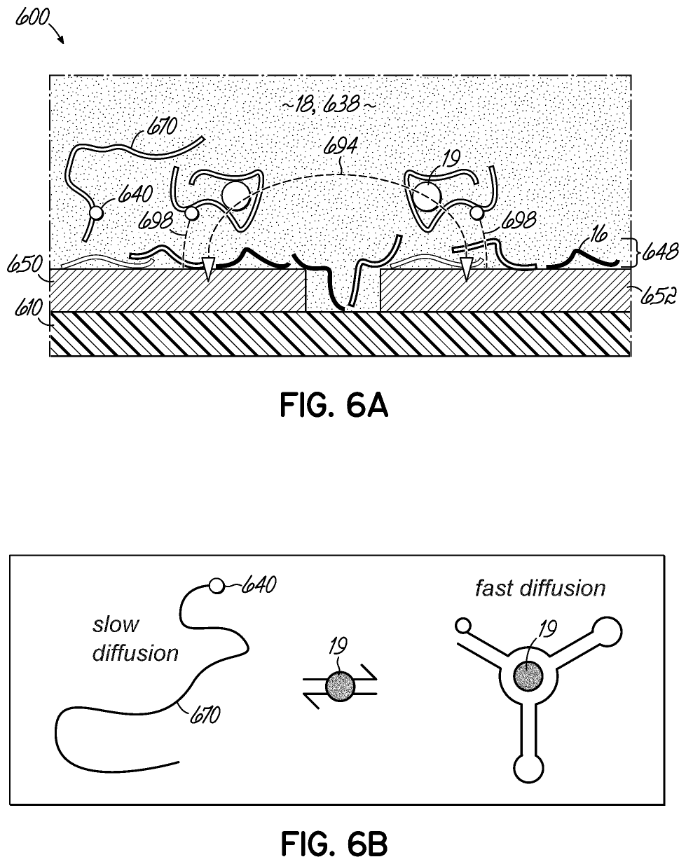

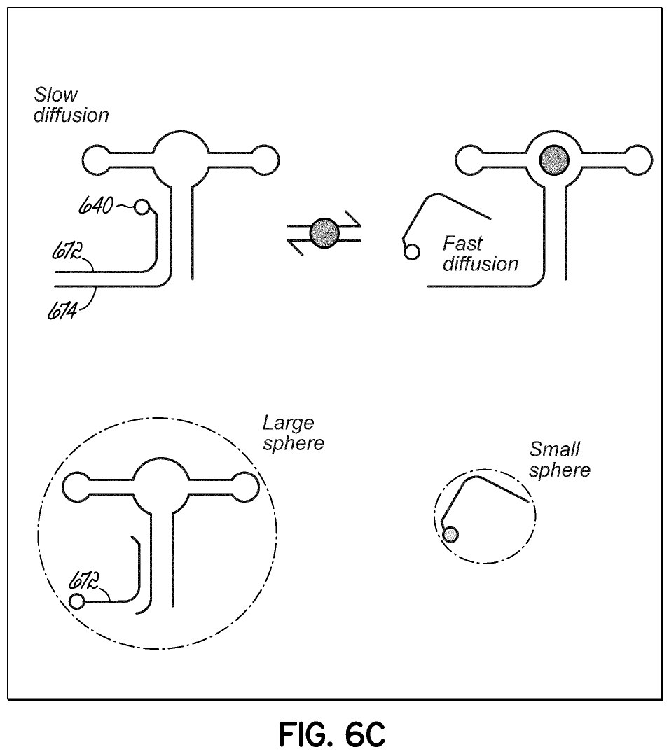

Turning now to FIGS. 6A-6C, where like numerals refer to like features: FIG. 6A shows a portion of a device 600 that includes a substrate 610, at least first and second electrodes 650, 652, a passivation layer 648 including endogenous solutes 16, a sensor fluid 18 (which, in the embodiment illustrated in FIG. 6A is inside an optional hydrogel 638), a plurality of aptamers 670 having redox tags 640 (free in solution), and a diffusion or iontophoretic pathway 694. Though not part of the device, analytes 19 are also depicted as present in the sensor fluid of the device. FIG. 6A also schematically depicts electron transfer that can occur between redox tags 640 and the first and second electrodes 650, 652. As can be seen in FIG. 6A, as a non-limiting example, electron transfer 698 from the redox tags 640 in an increased amount, or frequency, or rate, when analyte 19 is bound to the aptamer 670. For example, when analyte is bound to aptamer, the hydrodynamic radius or size of the aptamer is smaller and therefore providing a faster diffusion coefficient, which results in the redox tag being more available for electron transfer with the electrodes (such a version will be discussed in greater detail below with respect to FIG. 6B); or, for example, when analyte is not bound to aptamer, the redox tag is not available—or is less available—to the electrode, due to, for example, a conformation of the aptamer that hinders or prevents such transfer when not bound to analyte; conversely, when aptamer binds analyte, the conformation of aptamer may change in a manner that positions the redox tag for electron transfer. In various embodiments, aptamer binding to analyte can provide changes in electron transfer and redox current (compared to baseline transfer and current—i.e., transfer/current in the absence of analyte binding) of greater than 5%, greater than 10%, greater than 20%, greater than 50%, greater than 100%, or greater than 200%. For the embodiments illustrated in FIGS. 6A-6C, non-limiting examples of electrical measurement techniques may include voltammetry, square wave voltammetry, amperometry, chronoamperometry, coulometry, chronocoulometry, with a preferred embodiment being amperometry.

FIG. 6B schematically depicts another example of an aptamer 670 with attached redox tag 640 (that differs from the aptamer 670/redox tag 640 schematically shown in FIG. 6A). The embodiment of the aptamer in FIG. 6B is designed such that the redox tag 640 is less available for electron transfer with the electrodes 650, 652 in the absence of analyte binding to aptamer (left side of FIG. 6B), because of a longer diffusion time between the first and second electrodes 650, 652 where the analyte can undergo redox recycling (e.g., one electrode is a reducing electrode, one electrode is an oxidizing electrode). However, when analyte 19 binds to the aptamer 670 (right side of FIG. 6B), the hydrodynamic radius or size of the aptamer is smaller and therefore providing a faster diffusion coefficient, and therefore redox tag 640 is more available for electron transfer with the first and second electrodes 650, 652. The binding of analyte 19 transforms the aptamer 670 between a long unfolded aptamer 670 (in the absence of analyte 19 binding) and an aptamer 670 with three stems when analyte 19 binds to aptamer 670.

As described above, with respect to FIG. 6A, a non-limiting example of an environment within a device 600 may include an optional hydrogel. In such an embodiment, the hydrogel 638 (such as agar or polyacrylamide) is added to further distinguish diffusion times between aptamers 670 bound to analyte 19 and aptamers 670 not bound to analyte. This is because the hydrogel 638 creates a more tortuous and size-selective diffusion pathway than a pure fluid would by itself. For example, an aptamer 670 that fully dissociates could be modified to have a significant change in hydrodynamic radius (R), which changes its diffusion coefficient (D) according to D=kT/(6πηR). This equation is for diffusion in pure solution; a dense hydrogel 638 can be added to further distinguish the diffusion of the unfolded aptamer vs. the folded aptamer. The resulting current between the redox recycling electrodes is proportional as I∝DC/z, where C is the concentration of the aptamer 670 and z the electrode-to-electrode distance. With respect to changes in signal gain, the diffusion length of oglionucleotides (aptamers) varies with length to the ˜0.6^{th} power, and a 15 kDa protein that is globular/unfolded can have a change in R of 2.15/3.65.

FIG. 6C schematically depicts yet another example of an aptamer 670 with attached redox tag 640 (that differs from the aptamer 670/redox tag 640 schematically shown in FIGS. 6A and 6B). The embodiment of the aptamer in FIG. 6C is designed with two aptamer portions: a signaling aptamer 672 and an anchor aptamer 674. A redox tag 640 is associated with (such as by being attached to) the signaling aptamer 672. The anchor aptamer 674 includes a portion that has affinity for, and thus can bind, analyte 19. When analyte 19 is not bound to the anchor aptamer 674 (left side of FIG. 6C), the signaling aptamer 672 remains associated with the anchor aptamer 674, and so the redox tag 640 on signaling aptamer 672 is less available for electron transfer with the first and second electrode 650, 652 (as the combined signaling and anchor aptamers 672, 674 will exhibit slower diffusion in sensor solution and hydrogel). However, once the anchor aptamer 674 binds to analyte 19 (right side of FIG. 6C), signaling aptamer 672 is released from anchor aptamer 674, and the redox tag 640 becomes more available for electron transfer with the first and second electrodes 650, 652 (as the liberated signaling aptamer 672 will exhibit more rapid diffusion in sensor solution and hydrogel). Further, while the embodiment shown in FIG. 6C depicts analyte 19 binding to anchor aptamer 674 and redox tag 640 on signaling aptamer 672, in an alternate embodiment analyte binding may occur with signaling aptamer (signaling aptamer having redox tag), and binding of analyte to signaling aptamer may serve to release signaling aptamer from anchor aptamer (such as by change in conformation of signaling aptamer).

It will be recognized that when the device shown in FIG. 6A uses the embodiment of aptamers of FIG. 6C, it will include a plurality of signaling aptamers 672, and a plurality of anchor aptamers 674. As described above, each anchor aptamer of the plurality of anchor aptamers is adapted to bind to analyte. In one embodiment, each signaling aptamer of a majority of the plurality of signaling aptamers is bound to a respective anchor aptamer when a majority of anchor aptamers are not bound to any analyte. (This may occur, for example, prior to the introduction of any analyte.) Once analyte is introduced (such as when a sample fluid is introduced into the device—e.g., by being introduced into the sensor fluid of the device), at least a subset of anchor aptamers from the plurality of anchor aptamers then binds to analyte. When this occurs, a subset of signaling aptamers (from the total plurality of aptamers) dissociates from the anchor aptamers—and the redox tag becomes more available for electron transfer with the electrode.

With further reference to FIG. 6C, in addition to changes in diffusion coefficient, the larger the effective sphere for the aptamer the less likely it will experience electron transfer with an electrode (with a first principles estimation based on the inverse of sphere area, proportional to 1/R\hat{ }2). This example is simply to show that two factors can be at play for embodiments of the present invention, both distance of the redox tag to the electrode and diffusion time to/from the electrode. This diffusion time to an electrode applies other embodiments as well, where for example with a chronoamperometric response for an aptamer the total current baseline could remain higher or reach baseline more quickly as diffusion coefficient for the aptamers increases. This diffusion time to an electrode may also impact interrogation methods such as square wave voltammetry, as aptamer that is near the electrode can contribute to the signal as well if it is able to diffuse to the electrode during each square window (during each voltage pulse that is applied). The first and second electrodes 650 and 652 can be closely spaced via interdigitation or other suitable technique, and, in such an embodiment, may be within less than 50 μm, less than 10 μm, less than 2 μm, or less than 0.4 μm distant of each other.

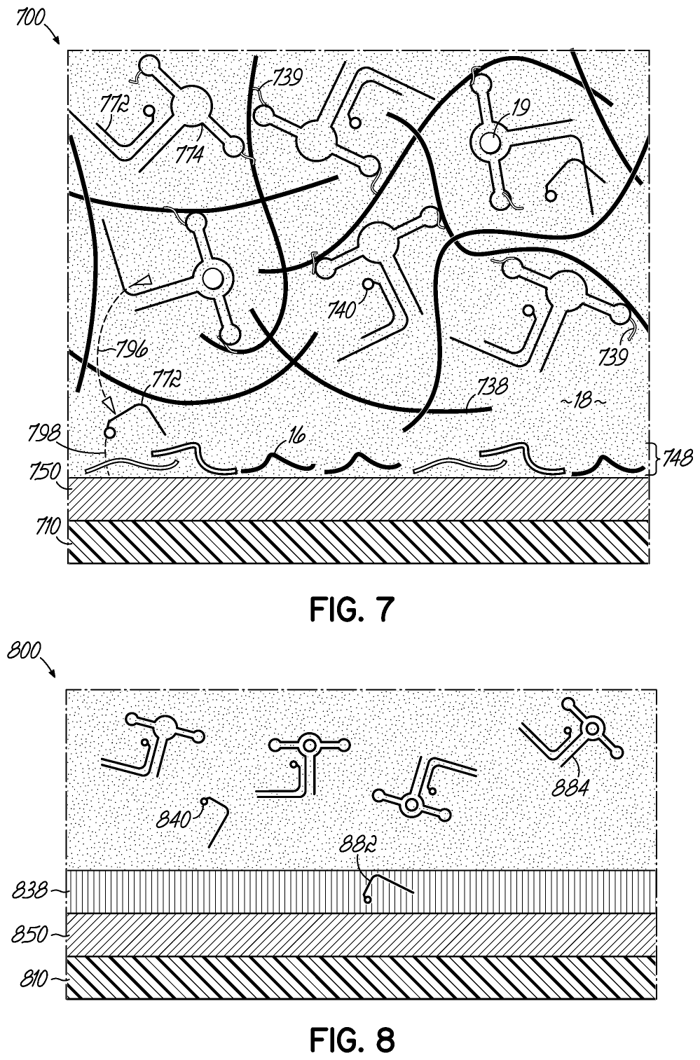

With reference to FIG. 7 where like numerals refer to like features, another embodiment in accordance with aspects of the present invention is shown. As can be seen in FIG. 7, a portion of a device 700 is shown, and includes a substrate 710, at least one electrode 750, a passivation layer 748 including endogenous solutes 16, a sensor fluid 18, a plurality of aptamers having redox tags 740 (free in the sensor solution), and a poorly-mobile or non-mobile material 738 in the sensor fluid 18. Though not part of the device, analytes 19 are also depicted as present in the sensor fluid of the device.

The aptamers/redox tags component of the embodiment of FIG. 7 is similar to that shown in FIGS. 5C and 6C, and includes two aptamer portions: a signaling aptamer 772 and an anchor aptamer 774. A redox tag 740 is associated with (such as by being attached to) the signaling aptamer 772. The anchor aptamer 774 includes a portion that has affinity for, and thus can bind, analyte 19. As can be seen in FIG. 7, the anchor aptamer 774 is immobilized via linkage 739 to the poorly or non-mobile material 738. The poorly-mobile or non-mobile material 738 may comprise various materials, such as a hydrogel. In one non-limiting example, the material 738 could be a hydrogel such as polyacrylamide and the linker be a molecule such as acrydite that is attached to the anchor aptamer at a terminal end or other location. In an alternate embodiment, the anchor aptamer could be cross-linked with other anchor aptamers or the anchor aptamer made so large (e.g., >100 kDa) such that it is effectively immobile in a dense hydrogel 738.

Still referring to FIG. 7, when analyte 19 is not bound to the anchor aptamer 774, the signaling aptamer 772 remains associated with the anchor aptamer 774, and so the redox tag 740 on signaling aptamer 772 is less available for electron transfer with the electrode 750 (because the combined signaling and anchor aptamers 772, 774 will be poorly-mobile or non-mobile in the sensor fluid due to anchor aptamer 774 being linked to material 738). However, once the anchor aptamer 774 binds to analyte 19, the signaling aptamer 772 is released from anchor aptamer 774 (as indicated by arrow 796), and the redox tag 740 becomes more available for electron transfer with the electrode 750 (because the liberated signaling aptamer 772 will exhibit more rapid diffusion in sensor solution as it is no longer complexed with the anchor aptamer 774 that is linked to poorly-mobile or non-mobile material 738). Further, while the embodiment shown in FIG. 7 depicts analyte 19 binding to anchor aptamer 774 and redox tag 740 on signaling aptamer 772, in an alternate embodiment analyte binding may occur with signaling aptamer (signaling aptamer having redox tag), and binding of analyte to signaling aptamer may serve to release signaling aptamer from anchor aptamer (such as by change in conformation of signaling aptamer).

It will be recognized that the device of the embodiment of FIG. 7 has a plurality of aptamers—and thus includes a plurality of signaling aptamers 772, and a plurality of anchor aptamers 774. As described above, each anchor aptamer of the plurality of anchor aptamers is adapted to bind to analyte. In one embodiment, each signaling aptamer of a majority of the plurality of signaling aptamers is bound to a respective anchor aptamer when a majority of anchor aptamers are not bound to any analyte. (This may occur, for example, prior to the introduction of any analyte.) Once analyte is introduced (such as when a sample fluid is introduced into the device—e.g., by being introduced into the sensor fluid of the device), at least a subset of anchor aptamers from the plurality of anchor aptamers then binds to analyte. When this occurs, a subset of signaling aptamers (from the total plurality of aptamers) dissociates from the anchor aptamers—and the redox tag becomes more available for electron transfer with the electrode.

With reference to FIG. 8, where like numerals refer to like features, another embodiment in accordance with aspects of the present invention is shown. As can be seen in FIG. 8, a portion of a device 800 is shown, and includes a substrate 810, at least one electrode 850, a membrane 838, a sensor fluid 18, a plurality of aptamers having redox tags 740 (free in the sensor fluid). Though not part of the device, analytes 19 are also depicted as present in the sensor fluid of the device. The aptamers/redox tags component of the embodiment of FIG. 8 is similar to that shown in FIGS. 5C, 6C, and 7, and includes two aptamer portions: a signaling aptamer 882 and an anchor aptamer 884. A redox tag 840 is associated with (such as by being attached to) the signaling aptamer 882. The anchor aptamer 884 includes a portion that has affinity for, and thus can bind, analyte 19. The membrane 838 exhibits selective permeability based on size, charge, or at least one solute property, and is able to pass a signaling aptamer 882 but not a signaling aptamer that is attached to a larger anchor aptamer 884. Thus, the membrane 838 impacts the availability of the redox couple 840 to the electrode 850. For example, a signaling aptamer could have a radius of 3 nm/2 nm in folded/unfolded states and an anchor aptamer have 27/7 nm in folded/unfolded state, creating a difference in size of ˜3-10× when a signaling aptamer is freed from an anchor aptamer. Nanofiltration membranes can provide is nM pore sizes, and ultrafiltration 10s to 100s nm pore sizes (PES, track-etch, and other materials), resulting in size selective permeability that would enable mainly only the signaling aptamer 882 to penetrate the hydrogel or membrane 838.

And so, still referring to FIG. 8, when analyte 19 is not bound to the anchor aptamer 884, the signaling aptamer 882 remains associated with the anchor aptamer 884, and so the redox tag 840 on signaling aptamer 882 is less available (or not available) for electron transfer with the electrode 850 (because the signaling aptamer 882 will be unable to cross membrane 838 due to being complexed with anchor aptamer 884). However, once the anchor aptamer 884 binds to analyte 19, the signaling aptamer 882 is released from anchor aptamer 884 and is able to pass through membrane 838, resulting in the redox tag 840 becoming available for electron transfer with the electrode 850. Further, while the embodiment shown in FIG. 8 depicts analyte 19 binding to anchor aptamer 884 and redox tag 840 on signaling aptamer 882, in an alternate embodiment analyte binding may occur with signaling aptamer (signaling aptamer having redox tag), and binding of analyte to signaling aptamer may serve to release signaling aptamer from anchor aptamer (such as by change in conformation of signaling aptamer).

As described above, one aspect of the present invention provides a device including a dissolvable material, and a plurality of aptamers disposed in and/or on the dissolvable material. The device may further include a volume (such as one that may be defined by one or more substrates) that is adapted to receive a sample fluid to be tested. The volume is in communicating relationship with the dissolvable material. And so, once sample fluid is introduced into the volume, the sample fluid will come into contact with the dissolvable material. This, in turn, releases one or more aptamers into the sample fluid. One or more aptamers of the plurality of aptamers (1) are capable of binding at least one analyte, and (2) have at least one redox tag attached thereto. And so, once released into the sample fluid, binding may occur, and the device further includes at least one electrode that is capable of detecting or measuring the at least one analyte through a change in electron transfer with the redox tags attached to the one or more aptamers.

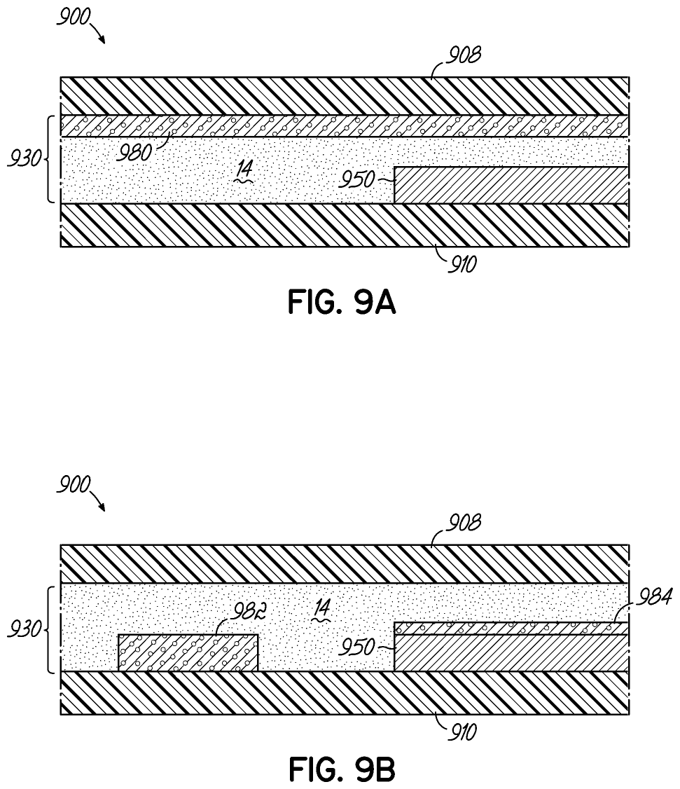

Embodiments of a portion of this type of device are shown schematically in FIGS. 9A and 9B. Turning first to FIG. 9A, a device 900 is shown as having a first substrate 908 and a second substrate 910. These first and second substrates 908, 910 define a volume 930 that is capable of receiving a sample fluid 14. A dissolvable material 980 (having a plurality of aptamers in, or on, or both in and on the material) is present in the volume 930—and in the embodiment of FIG. 9A is shown as positioned adjacent to the first substrate 908. In alternate embodiments, the dissolvable material may actually be the plurality of aptamers (for example, the plurality of aptamers could be positioned within the volume, and a substance that can dissolve nucleic acids, such as EDTA, could be added to the fluid prior to introduction of the fluid to the volume). The device then also includes a working electrode 950 positioned in the volume 930. In the embodiment shown in FIG. 9A the electrode 950 is shown adjacent to the second substrate 910. The portion of the device 900 shown in FIG. 9A (and the components shown schematically as part of that device) could be incorporated into other embodiments of devices disclosed herein. For example, device 900 could be part of a microneedle-type device—such as that shown in FIG. 1A and described above.

Although FIG. 9A shows first and second substrates 908, 910, alternate embodiments may include only a first substrate. The dissolvable material could then be adjacent, or on, or in, this first substrate, and the electrode could also be associated with the first substrate. Such an embodiment could be in the form of a test strip for finger prick blood, for example.

At least a portion of a surface of the device 900 may be coated with the dissolvable material 980 that contains the aptamer (and/or other solutes if desired, such as pH buffer). In one embodiment (see FIG. 9A), the dissolvable material 980 may be positioned (such as by being coated along the surface area of the first substrate 908 that serves to define (or partially define) the volume 930 that receives sample fluid 14 in the device 900. In various embodiments, varying percentages of the surface area may be covered with dissolvable material. For example, at least greater than 20%, greater than 50%, or greater than 90% of a surface (such as first substrate 908) may be covered. As can be seen in FIG. 9A, the dissolvable material 980 is disposed on first substrate 908 while working electrode 950 is disposed on a different substrate (the second substrate 910).

Alternatively, and referring now to FIG. 9B, dissolvable material 982 (including a plurality of aptamers therein and/or thereon; or where the plurality of aptamers may be the dissolvable material) may be positioned at a particular location (rather than being coated along percentage of surface area as described with respect to FIG. 9A). Additionally, as can be seen in FIG. 9B, the dissolvable material 982 is disposed on the same substrate (second substrate 910) as the working electrode 950. In the device illustrated in FIG. 9B, as sample fluids wicks into the device 900 (which can be as fast as seconds for glucose test strips as an example) material dissolves, such that the plurality of aptamers (with attached redox tags) are released into the fluid. And the plurality of aptamers then may progress toward a steady-state (or near-steady-state concentration) across the sample fluid 14 by the time fluid reaches working electrode 950.

In the particular embodiment shown in FIG. 9B, the dissolvable material 982 may not dissolve uniformly into the fluid, and if, for example, dissolvable material 982 were 5 mm distant from electrode 950, it could take 10s of minutes or even hours for concentration of the aptamer to equilibrate above the electrode 950 especially for high molecular weight aptamers. (This same issue would not be presented in the embodiment of FIG. 9A, because, as described above, the material 980 in that embodiment is coated along at least greater than 20%, greater than 50%, or greater than 90% of a surface (such as first substrate 908).

However, with further reference to FIG. 9B, if the dissolvable material 982 comprises a fractional area of a surface (such as surface 910), then the dissolvable material 982 could instead be designed to dissolve at a slow or constant rate, such that a more uniform concentration of aptamer is released into the sample fluid 14. For example, if the volume 930 were designed such that it required 2 seconds for sample fluid 14 to reach electrode 950, and the material 982 were 20 μm thick, then if material 982 dissolved at a rate of 10 μm per second, a more even distribution would be provided into sample fluid 14. Fluid velocity in the channel defined by volume 930 decreases with time and distance because the resistance to fluid flow is proportional to the channel length with sample fluid 14 in the volume 930. Thus, a uniform distribution of aptamer in the sample fluid would be maximized if the dissolution rate of material 982 remained proportional to the fluid velocity rate above material 982 as sample 14 is introduced into space 930.

A layer of material 982 with a variable dissolution rate with depth could add complexity and/or cost to the device. And so, if such were a concern, one could use the configuration shown in FIG. 9A where material 982 is substantially above the electrode 950, or for example, an alternative embodiment where material 982 is coated onto electrode 950 or onto material 984. For example, material 982 coated onto a surface of a 50 μm thick channel that is defined by volume 930 could have a length along the axis of flow of sample 14 that is 3 mm and electrode 950 could be 1 mm in length along that same axis and centered under material 982. If the sample 14 wicks into the device 900 in 1 second and the material 982 dissolves in 4 seconds time, then adjacent to electrode 950 the concentration of aptamer will therefore be more uniform. As a result, the present invention includes a material 982 that dissolves entirely with a time that is at least 4× slower than the time required for sample 14 to fully wick into the volume 930.

Uniform or partial coating of surfaces (such as first substrate 908 and/or second substrate 910) inside the device 900 can be achieved, for example, by simply wicking the material 980 into the device 900 as a fluid and drying it, or by screen printing, inkjet printing, or other printing of the material 980 onto a portion of first and/or second substrates 908, 910.

If the maximum distance between material 980 and sample fluid when introduced is small (such as a 10 micron high channel height between substrates 908, 910) then the time for the plurality of aptamers to reach greater than 90%, or even greater than 50% of its equilibrium concentration in the sample fluid within 100 μm of the electrode 950 could be as little as on the order of seconds or minutes. And for a channel height ranging from 5 μm to 1000 μm this time could be less than 20 minutes, less than 5 minutes, less than 2 minutes, less than 1 minute, less than 30 seconds, or less than 10 seconds. This enables a device that is diffusion rate limited for its response to respond with 90% or 50% accuracy within these same time scales.

Also effecting the ability of the device to rapidy reach an aptamer equilibrium that allows for accurate measurement is the dissolution rate of material 980 into the sample fluid. In various embodiments, the material could include aptamers along with aptamer dissolving solutes such as Tris and EDTA, or a water soluble binding material such as compressed saccharides which with small particle sizes and a dissolution rate of >1 mm/s, or a solid water soluble polymer or sugar film which would dissolve more slowly, or slow dissolving polymers such as poly(anhydride) or poly(ortho ester) in solid film, or compressed power which can provide much slower dissolution rates as little as mm's/day for solid films, or any options, mixtures, or materials that would provide complete dissolution rates of material within seconds to minutes. A more gradual dissolution rate may be preferable in some cases. For example, if it requires 2 seconds for sample fluid such as whole blood to wick into a device 900 then material 982 could be near the inlet and dissolve gradually over 2 seconds such that a uniform concentration of aptamer is provided to the sample fluid as it enters the device 900.

As described above, embodiments as shown in FIGS. 9A and 9B may also be applied to a simple single-use device (examples being a device 100 of FIG. 1A that is applied to the skin 12 and take a single measurement of an analyte within minutes of application of the device 100, or a test-strip device that used blood such as a blood finger prick). Regardless of format (test strip, microneedle, etc.) for single-use devices the time it requires to get measurement from the device is an important performance parameter. This time at which an accurate reading may be taken is referred to herein as a time point. This time point is defined as the time at which a completed measurement occurs (such that the device can give the information, e.g., a measurement, to a user) subsequent to sample fluid being introduced to the device. A device that is rapidly ready to use is also valuable for a continuous sensing device. As described above, with solution phase aptamers, there are at least two challenges that increase the time before the device is ready to provide an accurate measurement: (1) the aptamers and any other solutes required for sensing must be in or nearly in a steady-state concentration across the sample fluid because measurement signal is proportional to concentration of the aptamers and redox tags; (2) the thickness of the passivating layer or fouling layer on the electrode should not be rapidly changing during the measurement because otherwise electron transfer rates from redox tags are altered. Resolution of the first of these challenges has been discussed above. Resolution of the second is now discussed below.

With reference to FIG. 9B, the device 900 includes a passivating layer 984 on electrode 950. The passivating layer may need to quickly stabilize, or be pre-stabilized, to a constant, or near-constant, thickness, because if the thickness of the passivation layer changes over time, it will alter the electron transfer rate from redox tags to the electrode 950, which could cause measurement errors. In one embodiment, pre-stabilization could be achieved by coating the electrode 950 with exogenous molecules, such as mercaptohexanol or polyethylene glycol. Alternatively, the electrode could be soaked in a fluid having endogenous solutes—i.e., soaking the electrode in a fluid that shares characteristics with the sample fluid (e.g., if the sample fluid is blood, the electrode could be soaked in serum). In such an embodiment, the electrode 950 could be soaked for 10s of minutes or more to allow endogenous passivation of the electrode, and then drying of the electrode surface. In all of these-prestabilized embodiments, the material 984 could also include at least one preservative or other material that is water-soluble, such as trehalose, to enable dry storage of electrode 950 with a pre-stabilized surface.

If not pre-stabilized, then rapid stabilization during use of the device 900 could be achieved, for example, by coating electrode 950 with a material 984 such as dried blood. In such an embodiment, when the blood is rehydrated (due to presence of sample fluid) a very high solute concentration will be present initially, which will expedite the diffusion of passivating solutes to the electrode 950 surface and stabilize the thickness of the passivating layer (e.g., in a time as rapid as less than one minute) such that the thickness of the passivation layer 984 will changes by less than 50%, less than 20%, or less than 10% during measurement of the analyte. Other pre-stabilization or stabilization methods are possible, so long as they satisfy the performance requirements as described for embodiments of the present invention.

The various embodiments disclosed herein can be enabled to be user-calibrated, factory-calibrated, or calibration-free. User-calibration could for example require a pin-prick blood draw and running of a conventional assay to measure analyte concentration, and that concentration data entered into the software that runs the sensing device.

Factory-calibrated implies that the device requires calibration, but that the calibration is shelf-stable and stable for at least a portion of the use period of the device. Embodiments, such as those shown in FIG. 5B and FIG. 5C, could benefit from factory calibration if they are interrogated by square wave voltammetry, and if the passivation layer 548 thickness is kept fairly constant (e.g., using a mercaptohexanol passivation layer 548 or polyethylene glycol terminated passivation layer 548 that is resistant to fouling). In factory calibration, the device is tested with a sample fluid with a known concentration of analyte, and that information is then shipped along with the product in order to enable it to start its use with proper calibration.

Calibration-free operation is possible if one could eliminate the factors that could cause a sensor signal to drift or change. Considering the embodiments of FIGS. 6 and 9, the aptamer concentrations can be kept constant, and with a chronoamperometric measurement response the change in current vs. time will be dependent on diffusion coefficient of the signaling aptamer 672 vs. the anchor aptamer 674 and signaling aptamer 672 bound together. The diffusion coefficient will not change in a sample fluid such as interstitial fluid, and an overvoltage can be supplied to measure the chronoamperometric response even if thickness of the passivating layer 648 changes slightly. Simply, the chronamperometric response will measure the percentage of signaling aptamer 672 that is free from the anchor aptamer 674, which is directly related to the binding affinity of the analyte to the aptamers, hence enabling calibration-free operation because the concentrations of the signaling aptamers 672 and anchor aptamers 674 are known. Calibration-free operation is also possible using the constructs of FIG. 5D or FIG. 5E by measuring changes in electron transfer rates, peak position shifts, or ratios (not individual magnitudes) of two or more redox peaks from different redox tags 546, 548 (FIG. 5E).

### EXAMPLES

### Example 1

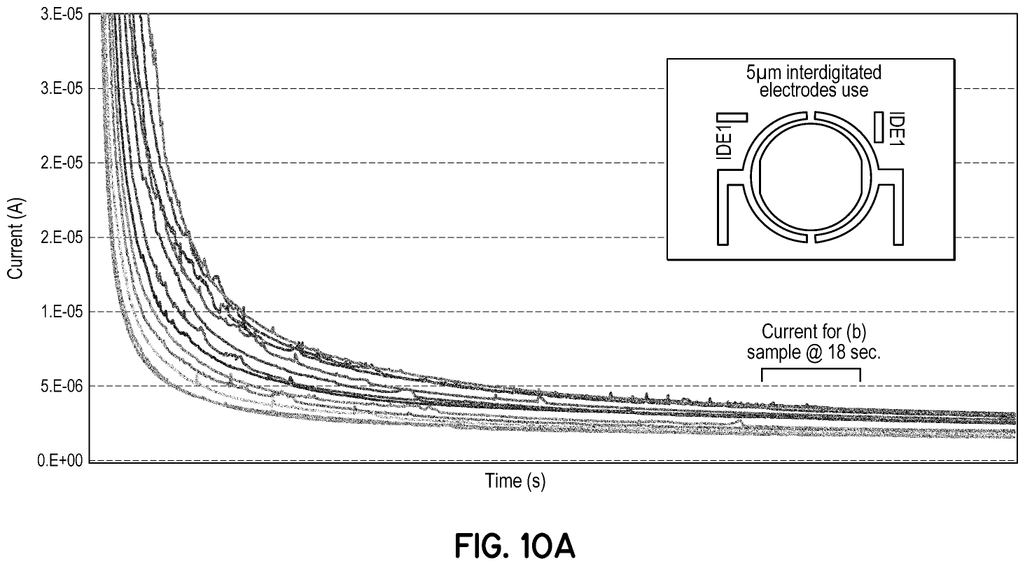

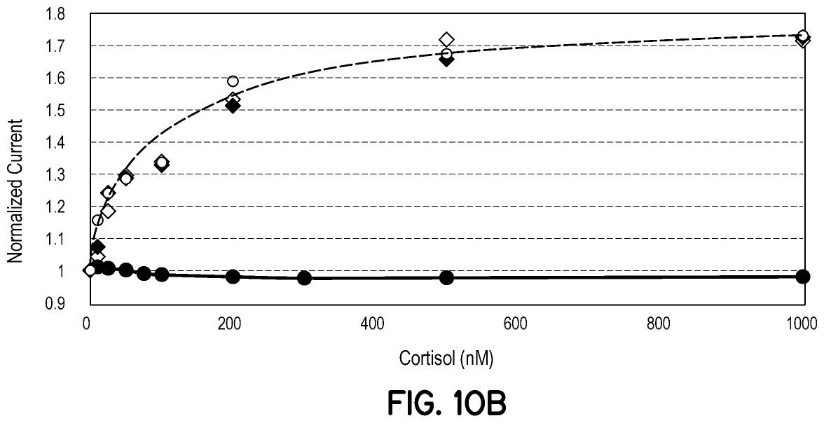

With reference to FIGS. 10A and 10B a cortisol binding aptamer was utilized in a manner similar to that taught in FIG. 6C, where the signaling aptamer 672 was tagged with methylene blue as a redox tag 640 with an aptamer sequence of GTCGTCCCGAGAG [SEQ ID NO.1] and where the anchor aptamer 674 with a sequence of ctctcgggacgacGCCCGCATGTTCCATGGATAGTCTTGACTAgtcgtccc [SEQ ID NO. 2]. Electrodes 650, 652 were gold interdigitated electrodes with a 5 μm spacing in between them. The gold electrodes were passivated with an exogenous molecule of mercaptohexanol. No hydrogel 638 was utilized in this experiment. The sensor solution was buffer solution with 5 μM of the aptamers 650, 652 in solution, and a reference electrode of platinum was used. The device 600 was measured amperometrically vs. a titration curve of cortisol as the analyte 19. The results are shown in FIG. 10A and FIG. 10B (open circles, open diamonds, and solid diamonds), and a control experiment with titration of simply adding more cortisol but without aptamer in solution is also shown in FIG. 10B (solid circles). The signal gain in Example 1 is as much as 70%, and if the anchor aptamer was made even larger or smaller the signal gain could be tuned to be as much as 200% or more as little as 5% based on the change in diffusion rate of the signaling aptamer to the electrode compared to the signaling aptamer when it is bound to the anchor aptamer. Signal gain is also measured above a baseline signal, and changing signaling aptamer concentration can therefore be used to tune the signal gain.

### Example 2

The experiment of Example 1 was repeated but instead of using mercaptohexanol passivation of the gold electrodes 650, 652, endogenous small molecule solutes found in blood or interstitial fluid were allowed to passivate the gold electrode 650, 652. It was found that without passivation background current was very high, but that both mercaptohexanol and endogeneous solutes were able to adequately reduce background current and enable sensor operation.

### Example 3

Sensors were tested with square wave voltammetry, and redox peaks via voltammetry were observable with 100 nM of aptamer. Higher aptamer concentrations only increase the amount of signal and 1 μM, and 10 μM and 100 μM of aptamer were tested as well. Generally, lower aptamer concentrations were preferred as they reduce device lag times as they require less concentration of analyte to create a change in sensor signal.

### Example 4

With respect to an embodiment of the present invention, commercial devices generally need to be shelf-stable. The present invention can benefit from several methods to promote shelf stability. In one embodiment, the device is stored wet, because DNA is storable in wet aqueous conditions for years such as storing in buffer solution. In another embodiment, the device is stored dry. The solutes in the sensor fluid can be stored dry in a suspending matrix. Non-limiting examples include a sugar such as trehalose, whole biofluid or solutes in a biofluid such as serum, by applying the sensor fluid with this suspending matrix, drying it, and then storing in a dry state. The solutes in the sensor fluid may also dried along with one or dissolution-promoting material such as TE buffer containing Tris buffer and ETDA chelating agent, such that the aptamer rapidly resolubilizes when a dry device is placed into sample fluid. With dry storage, an additional challenge is rewetting of the device without air-bubbles being trapped inside the device. For example, if the membrane became uniformly wet before water from the sample fluid penetrated into the device, the wet membrane could entrap air. Therefore, in one embodiment the present invention includes at least one portion of the membrane that is hydrophobic, by treating with a dilute fluoropolymer solution. In an alternative embodiment, the device includes at least one hydrophobic vent such as porous Teflon, to allow an escape route for any gas in the device as it wets initially with sample fluid. In an alternate embodiment, there is no gas in the device before it is wetted with sample fluid, achieved by storing the device in a vacuum state or by storing the device such that all gas in the device is replaced by a dissolvable solid such as a sugar. In yet another embodiment, initial wetting is directional, such as being from one side of the device initially as it is first placed into sample fluid, allowing air to escape out the initially unwetted portion of the membrane. In yet another alternate embodiment, the space normally occupied by the sensor fluid also contains at least one hydrogel such as agar or acrylate, that wets, potentially expands, and displaces gas from the device. This same hydrogel could be used to create a spacer between the membrane and the substrate carrying the electrodes, and the hydrogel and device alternately could be stored wet.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A device for detecting the presence of, or measuring the amount or concentration of, at least one analyte in a sample fluid, the device comprising:
a dissolvable material;
a plurality of aptamers disposed in and/or on the dissolvable material, or being the dissolvable material, wherein one or more aptamers of the plurality of aptamers:
(i) are capable of binding at least one analyte, and
(ii) have at least one redox tag attached thereto; and
at least one electrode that is capable of detecting or measuring the at least one analyte through a change in electron transfer with the redox tags attached to the one or more aptamers.

2. The device of claim 1, further comprising a volume that is adapted to receive a sample fluid, wherein the volume is in communicating relationship with the dissolvable material.

3. The device of claim 2, further comprising a first substrate, and wherein the volume is defined by the first substrate.

4. The device of claim 3, further comprising a second substrate, and wherein the volume is defined by the first and second substrates.

5. The device of claim 3, wherein the dissolvable material is positioned within the volume and is adjacent the first substrate.

6. The device of claim 4, wherein the dissolvable material is positioned within the volume and is adjacent the first substrate or the second substrate.

7. The device of claim 6 wherein the dissolvable material is positioned on a percentage of a surface area of the first substrate or the second substrate, wherein the percentage is selected from the group consisting of greater than 20%, greater than 50%, and greater than 90%.

8. The device of claim 2, wherein the volume provides a fluid path for the sample fluid, wherein the fluid path comprises an entry point for the sample fluid, wherein the electrode is positioned in the fluid path, and wherein the fluid path comprises a distance between the entry point and the electrode.

9. The device of claim 8, wherein the electrode is capable of providing information that correlates to the detection or measurement of the at least one analyte, and the provision of the information occurs at a defined time point, wherein the time point is subsequent to introduction of the sample fluid into the fluid path.

10. The device of claim 9, wherein the time point is selected from the group consisting of less than 20 minutes after introduction of the sample fluid into the fluid path, less than 5 minutes after introduction of the sample fluid into the fluid path, less than 2 minutes after introduction of the sample fluid into the fluid path, less than 1 minute after introduction of the sample fluid into the fluid path, less than 30 seconds after introduction of the sample fluid into the fluid path, and less than 10 seconds after introduction of the sample fluid into the fluid path.

11. The device of claim 9, wherein the time point occurs when the concentration of aptamer reaches >50% of its equilibrium concentration in the sample fluid within 100 μm distance of the electrode.

12. The device of claim 9, wherein the time point occurs when the concentration of aptamer reaches >90% of its equilibrium concentration in the sample fluid within 100 μm distance of the electrode.

13. The device of claim 10, wherein the information that correlates to the detection or measurement of at least one analyte is information that correlates to measurement of at least one analyte, and wherein the measurement of analyte is greater than 50% of a measurement of analyte if the aptamer were allowed to diffuse uniformly in the sample fluid.

14. The device of claim 10, wherein the information that correlates to the detection or measurement of at least one analyte is information that correlates to measurement of at least one analyte, and wherein the measurement is greater than 90% of a measurement if the aptamer were allowed to diffuse uniformly in the sample fluid.

15. The device of claim 1, further comprising a solute capable of dissolving the dissolvable material.

16. The device of claim 1, wherein the electrode is pre-stabilized with a passivating layer.

17. The device of claim 16, wherein the passivating layer is comprised of primarily exogenous molecules.

18. The device of claim 16, wherein the passivating layer is comprised of primarily molecules that are also endogenous to a sample fluid.

19. The device of claim 1, wherein the electrode is coated with at least one stabilizing material that is dissolvable in a sample fluid.

20. The device of claim 1, further comprising a passivating layer on the electrode, wherein the passivating layer has a thickness that, after being in contact with a sample fluid for one minute or less, that changes by a percentage chosen from less than 50%, less than 20%, and less than 10%.

21. The device of claim 20, wherein the change in the percentage is less than 50%, less than 20% or less than 10% during detecting or measuring the at least one analyte through a change in electron transfer with the redox tags attached to the one or more aptamers.

22. The device of claim 1, further comprising a sensor fluid, and further comprising at least one membrane between the sensor fluid and the electrode.

23. The device of claim 22, wherein the membrane has at least one portion of the membrane that is adequately hydrophobic to vent gas.

24. The device of claim 22, wherein the membrane and electrode are separated by a fluid-incorporable material.

25. The device of claim 24, wherein the fluid-incorporable material is the dissolvable material.

26. The device of claim 1, further comprising a plurality of microneedles.

27. The device of claim 1, wherein the dissolvable material dissolves at a rate proportional to the rate of velocity of the sample fluid above the dissolvable material as the sample fluid is introduced into the device.

28. The device of claim 1, further comprising a first time for sample fluid to fully fill the device, a second time for the dissolvable material to dissolve in the sample fluid, and wherein the second time is at least four times greater than the first time.

29. A method of detecting the presence of, or measuring the concentration or amount of, an analyte in a sample fluid, the method comprising:
introducing a sample fluid to a volume having a dissolvable material and at least one electrode therein, wherein the dissolvable material comprises a plurality of aptamers having a plurality of redox tags attached thereto; and
measuring the analyte based on a change in electron transfer between one or more redox tags of the plurality of redox tags and the electrode, wherein measuring the analyte occurs at a time point, wherein the time point is subsequent to introduction of the sample fluid into the fluid path.

30. The method of claim 29, wherein measuring the analyte is performed at a time point subsequent to introduction of the sample fluid into the volume.

31. The method of claim 30, wherein the time point is selected from the group consisting of less than 20 minutes, less than 5 minutes, less than 2 minutes, less than 1 minute, less than 30 seconds , and less than 10 seconds.

32. The method of claim 30, wherein the time point occurs when the concentration of aptamer reaches >50% of its equilibrium concentration in the sample fluid within 100 μm distance of the electrode.

33. The method of claim 30, wherein and the time point occurs when the concentration of aptamer reaches >90% of its equilibrium concentration in the sample fluid within 100 μm distance of the electrode.

34. The method of claim 30, wherein the measurement is greater than 50% of a measurement if the aptamer were allowed to diffuse uniformly in the sample fluid, and wherein the time point is selected from the group consisting of less than 20 minutes, 5 minutes, 2 minutes, 1 minute, 30 seconds, and 10 seconds.

35. The method of claim 30, wherein the measurement is greater than 90% of a measurement if the aptamer were allowed to diffuse uniformly in the sample fluid, and wherein the time point is selected from the group consisting of less than 20 minutes, 5 minutes, 2 minutes, 1 minute, 30 seconds, and 10 seconds.


SEQUENCE LISTING (2 sequences)

SEQ ID NO: 1 - DNA, 13 bp, Artificial Sequence
  Synthetic
gtcgtcccgagag

SEQ ID NO: 2 - DNA, 51 bp, Artificial Sequence
  Synthetic
ctctcgggacgacgcccgcatgttccatggatagtcttgactagtcgtccc